(19) United States
(12) Patent Application Publication
(10) Pub. No.: US 2026/0007463 A1
(43) Pub. Date: Jan. 8, 2026

(54) SYSTEMS, DEVICES, COMPONENTS AND METHODS FOR NEAR-FIELD DIFFERENTIAL COMPLEX IMPEDANCE MEASUREMENTS IN INTRACARDIAC PULSED FIELD ABLATION CATHETERS

(71) Applicant: Cortex, Inc., Menlo Park, CA (US)

(72) Inventors: Peter Ruppersberg, Blonay (CH); Philip Haeusser, Muenchen (DE)

(21) Appl. No.: 19/261,337

(22) Filed: Jul. 7, 2025

Related U.S. Application Data
(60) Provisional application No. 63/668,092, filed on Jul. 5, 2024.

(51) Int. Cl. A61B 18/14; A61B 18/00

(52) U.S. Cl. CPC A61B 18/1492; A61B 2018/00351; A61B 2018/00577; A61B 2018/0072

(57) ABSTRACT

An intracardiac pulse field ablation (PFA) system comprising a catheter, a constant current AC signal generator, and at least one computing device, wherein the PFA catheter comprises at least a first constant AC current injection electrode, second and third sensing or recording electrodes, and a fourth ablation electrode, the PFA catheter and the electrodes thereof being configured to be operably connected to the constant current AC signal generator, and at least one computing device, the first electrode being a signal injection electrode configured to inject constant current AC signals into or near the patient's cardiac tissue, the second and third electrodes being recording or sensing electrodes configured and located sufficiently close to the first electrode to permit differential sensing and measurement of complex electrical impedance signals, including the phase and amplitude thereof, resulting from injection of the constant current AC signals into the cardiac tissue from the first electrode.

### CROSS-REFERENCE TO RELATED APPLICATIONS

The present application claims priority to U.S. Provisional Patent Application No. 63/668,092, filed Jul. 5, 2024, the entire disclosure of which is incorporated herein in its entirety.

### TECHNICAL FIELD

Various embodiments described and disclosed herein relate to the field of medicine generally, and more particularly to methods, systems, devices, and components configured to measure complex impedances in a patient's heart, as for example during pulsed field ablation procedures.

### BACKGROUND

Radiofrequency (RF) ablation is commonly employed to treat various forms of cardiac arrhythmias in patients. In some forms of cardiac arrhythmias, however, such as cardiac atrial tachycardias (AT), intra-atrial re-entry arrythmias, ventricular tachycardia, (VT), and atrial fibrillation (AF), and so on, accurate and efficacious cardiac ablation can pose a major challenge. This stems in part from the limitations of conventional fluoroscopic imaging techniques and conventional catheter-based electrophysiological (EP) mapping, and associated intra-body positioning and navigation techniques, that are used to determine the three-dimensional (3D) locations of arrhythmogenic cardiac substrates inside a patient's heart that are ablation targets.

The use of fluoroscopy for such navigation and positioning purposes can be problematic for several reasons. Fluoroscopy-guided catheter navigation is imprecise, time-consuming, and generally requires multiple different views to estimate with some degree of precision the 3D location of an ablation catheter. Moreover, using fluoroscopy, an ablation catheter often cannot be accurately and precisely returned to a previously mapped site. Fluoroscopy also exposes the patient and health care providers to radiation. More recent and improved non-fluoroscopic mapping systems have thus been created, and have enabled physicians to overcome some of the limitations of conventional fluoroscopic mapping systems. Some of these new and improved systems can provide higher mapping resolution, 3D spatial localization, and relatively rapid acquisition of cardiac activation maps.

Examples of such new systems include the CARTO, Ensite NavX, Rythmia, Affera, Acutus, NavX and CardioNXT systems. The CARTO, Ensite NavX, Rythmia, and Affera mapping, navigation and positioning systems utilize largely magnetic-based technologies, while the Acutus, NavX and CardioNXT systems utilize primarily impedance-based technologies.

These more recent navigation, positioning and mapping systems are generally quite complicated from a technical standpoint, can require the use of expensive specialized catheters along with complex and expensive external hardware, and may be complicated or cumbersome to deploy and use. Moreover, the navigation and positioning accuracy of ablation and EP mapping catheters inside a patient's heart using some of these more recent systems can be marginal, or at least not as precise as some physicians would otherwise desire. See “Physician-controlled costs: The choice of equipment used for atrial fibrillation ablation” to Winkle et al., J Interv Card Electrophysiol (2013), 36:157-165, DOI 10.1007/s10840-013-9782-x.

In addition, such as in pulsed field ablation (PFA) procedures, it can be difficult to measure and accurately modulate and control the amount of energy delivered to cardiac tissue so as to ablate errant electrical pathways associated with cardiac arrythmias, which is typically accomplished by measuring ohmic impedance in and around ablation catheter electrodes. For example, either too much energy can be delivered to the cardiac tissue, resulting in excessive and undesired damage to the cardiac tissue, or too little energy can be delivered to the cardiac tissue, resulting in an effective ablation procedure that does not successfully or sufficiently ablate the cardiac tissue to the degree required to destroy or disrupt the errant or undesired electrical pathways associated with cardiac arrythmias. It is estimated that at the present time approximately one quarter of all PFA procedures result in unsuccessful or insufficient ablation of cardiac tissue.

What is needed are improved, less complicated, faster, more accurate, and less expensive means and methods of mapping, positioning, and navigating ablation and electrophysiological (EP) mapping catheters inside a patient's heart and other internal organs. Achieving such goals would, by way of example, enable cardiac ablation procedures to be carried out more quickly, less expensively, and with greater locational precision, and would result in higher rates of success in treating cardiac rhythm disorders such as AF.

What is also needed are improved means of accurately sensing and measuring the impedance of cardiac tissue that has been or is being treated using pulsed field ablation (PFA) techniques to improve the results obtained in PFA procedures.

### SUMMARY

In Example 1, an intracardiac pulse field ablation (PFA) system comprising a catheter configured to ablate, and measure near-field complex electrical impedance of, cardiac tissue inside a patient's heart, a data acquisition, recording or measurement device, a constant current AC signal generator, and at least one computing device, wherein the PFA catheter comprises a proximal end and a distal end, a catheter body located between the proximal end and the distal end, and a plurality of electrodes including at least first, second and third electrodes, the PFA catheter and the electrodes thereof being configured to be operably connected to the data acquisition, recording or measurement device, the constant current AC signal generator, and at least one computing device, the first, second, third and fourth electrodes being located near or in the direction of the distal end, the first electrode being operable as a signal injection electrode configured to inject constant current AC signals into or near the patient's cardiac tissue, the second and third electrodes being operable as recording or sensing electrodes configured and located sufficiently close to the first electrode to permit differential sensing and measurement of complex electrical impedance signals, including the phase and amplitude thereof, resulting from injection of the constant current AC signals into the cardiac tissue from the first electrode, wherein one or more of the plurality of electrodes are further configured to deliver PFA energy into, and to ablate, the patient's cardiac tissue.

In Example 2, the intracardiac PFA system of Example 1, wherein electrical signals corresponding to transmitted controlled constant current AC signals sensed by the second and third electrodes as complex electrical impedance electrical signals are provided or relayed to the data acquisition, recording, or measurement device.

In Example 3, the intracardiac PFA system of Example 1, wherein the data acquisition, recording, or measurement devices are configured to relay the sensed electrical signals to the at least one computing device as sensed electrical signal values.

In Example 4, the intracardiac PFA system of Example 3, wherein the at least one computing device comprises at least one non-transitory computer readable medium configured to store instructions executable by at least one processor to permit detection or display to a user of changes in the amplitude and phase of the sensed electrical signals or values that correspond to a condition or state of the cardiac tissue that has been subjected to the PFA energy.

In Example 5. The intracardiac PFA system of Example 4, wherein the detected or displayed condition or state of the cardiac tissue is live tissue, dead tissue, or a combination of live and dead tissue.

In Example 6, the intracardiac PFA system of Example 4, wherein the detected or displayed condition or state of the cardiac tissue is reversibly electroporated tissue, irreversibly electroporated tissue, or a combination of reversibly electroporated tissue and irreversibly electroporated tissue.

In Example 7, the intracardiac PFA system of Example 4, wherein changes in the amplitude and phase of the sensed electrical signals or values are based upon comparisons between sensed electrical signals or values derived from the second electrode and sensed electrical signals or values derived from the third electrode.

In Example 8, the intracardiac PFA system of Example 1, wherein a distance between the first electrode and the second electrode, or between the first electrode and the third electrode, ranges between about 0.25 cm and about 4 cm.

In Example 9, the intracardiac PFA system of Example 1, wherein a distance between the first electrode and the second electrode, or between the first electrode and the third electrode, ranges between about 0.5 cm and about 2 cm.

In Example 10, the intracardiac PFA system of Example 1, wherein the constant current AC signals have a frequency ranging between about 10 kHz and about 500 KHz.

In Example 11, the intracardiac PFA system of Example 1, wherein the constant current AC signals have a frequency ranging between about 50 kHz and about 150 KHz.

In Example 12, the intracardiac PFA system of Example 1, wherein the constant current AC signals have a current ranging between about 0.4 mA and about 3 mA.

In Example 13, the intracardiac PFA system of Example 1, wherein the constant current AC signals have a current ranging between about 0.5 mA and about 1.5 mA.

In Example 14, the intracardiac PFA system of Example 1, wherein one or more of the polarity and functionality of at least one of the current injection electrode, the sensing electrodes, and the ablation electrode can be interchanged or switched.

In Example 15, the intracardiac PFA system of Example 1, further comprising a PFA generator configured to controllably provide pulsed field energy to the fourth ablation electrode.

In Example 16, an intracardiac method of sensing, recording and analyzing complex electrical impedance signals using an intracardiac pulse field ablation (PFA) catheter located inside a patient's heart, the PFA catheter comprising a proximal end and a distal end, a catheter body located between the proximal end and the distal end, and a plurality of electrodes including at least first, second and third electrodes, the PFA catheter and the electrodes thereof being configured to be operably connected to a data acquisition, recording or measurement device, a constant current AC signal generator, and at least one computing device, the first, second, third and fourth electrodes being located near or in the direction of the distal end, the first electrode being operable as a signal injection electrode configured to inject constant current

AC signals into or near the patient's cardiac tissue, the second and third electrodes being operable as recording or sensing electrodes configured and located sufficiently close to the first electrode to permit differential sensing and measurement of complex electrical impedance signals, including the phase and amplitude thereof, resulting from injection of the constant current AC signals into the cardiac tissue from the first electrode, wherein one or more of the plurality of electrodes are further configured to deliver PFA energy into, and to ablate, the patient's cardiac tissue, the method comprising: (a) positioning the PFA catheter inside the patient's heart so that the fourth ablation electrode is located near a target for cardiac tissue ablation; (b) using the fourth ablation electrode, applying pulsed field ablation energy to the target cardiac tissue; (c) using the first current injection electrode, injecting constant current AC signals into or near the target cardiac tissue; (d) using the second and third recording or sensing electrodes, differentially sensing and measuring complex electrical impedance signals resulting from injection of the constant current AC signals into the cardiac tissue from the first electrode; and (e) relaying the differentially sensed complex electrical impedance signals to the at least one computing device as sensed electrical signal values.

In Example 17, the method of Example 16, wherein the at least one computing device comprises at least one non-transitory computer readable medium configured to store instructions executable by at least one processor to permit detection or display to a user changes in the amplitude and phase of the sensed electrical signals or values that correspond to a condition or state of the cardiac tissue that has been subjected to the PFA energy, and further comprising at least one of detecting and displaying to the user changes in the amplitude and phase of the sensed electrical signals or values that correspond to a condition or state of the cardiac tissue that has been subjected to the PFA energy.

In Example 18, the method of Example 17, wherein changes in the amplitude and phase of the sensed electrical signals or values are based upon comparisons between sensed electrical signals or values derived from the second electrode and sensed electrical signals or values derived from the third electrode.

In Example 19, the method of Example 18, wherein the constant current AC signals have a frequency ranging between about 50 kHz and about 150 KHz.

In Example 20, the method of Example 19, wherein the constant current AC signals have a current ranging between about 0.5 mA and about 1.5 mA. While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a block diagram of a combined cardiac electrophysiological mapping (EP), pacing and ablation system, in accordance with embodiments of the disclosure;

FIG. 2 is a block diagram of a computer system for use in the EP, pacing and ablation system of FIG. 1;

FIG. 3 is a block diagram of a medical device or catheter controlled constant current navigation, positioning and/or imaging system 100, in accordance with embodiments of the disclosure;

FIG. 4 is a flow diagram corresponding to one embodiment of a method of navigating, positioning, and/or imaging a medical device or catheter inside a human body, in accordance with embodiments of the disclosure;

FIG. 5 is a schematic illustration of a linear point ablation catheter used in a Near-Field Differential Complex Impedance (NFDCI) system, in accordance with embodiments of the present disclosure;

FIG. 6 is an illustration demonstrating the working principles of the NFDCI system, in accordance with embodiments of the present disclosure; and

FIG. 7 is a schematic illustration of the operation of the NFDCI system utilizing a multi-splined pulsed field ablation catheter, in accordance with embodiments of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

Described herein are various embodiments of systems, devices, components and methods for one or more of EP mapping, and/or navigating or determining a position of a medical device such as an ablation and/or EP mapping catheter, or imaging the medical device, inside a patient's body (e.g., inside the patient's heart, stomach, brain or other internal organ, passageway, or other internal portion of the body). Also described and disclosed herein are various embodiments of pulsed field ablation near-field differential complex impedance measurement systems, devices, components and methods.

Various embodiments described and disclosed herein relate to systems, devices, components and methods for discovering with enhanced precision the location(s) of the source(s) of different types of cardiac rhythm disorders and irregularities. Such cardiac rhythm disorders and irregularities, include, but are not limited to, arrhythmias, atrial fibrillation (AF or A-fib), atrial tachycardia, atrial flutter, paroxysmal fibrillation, paroxysmal flutter, persistent fibrillation, ventricular fibrillation (V-fib), ventricular tachycardia, atrial tachycardia (A-tach), ventricular tachycardia (V-tach), supraventricular tachycardia (SVT), paroxysmal supraventricular tachycardia (PSVT), Wolff-Parkinson-White syndrome, bradycardia, sinus bradycardia, ectopic atrial bradycardia, junctional bradycardia, heart blocks, atrioventricular block, idioventricular rhythm, areas of fibrosis, breakthrough points, focus points, re-entry points, premature atrial contractions (PACs), premature ventricular contractions (PVCs), and other types of cardiac rhythm disorders and irregularities.

Various embodiments of electrographic flow (EGF) techniques, methods, systems, devices, and components may be employed efficaciously in conjunction with the controlled constant current navigation, positioning and imaging systems, devices, components and methods disclosed and described herein.

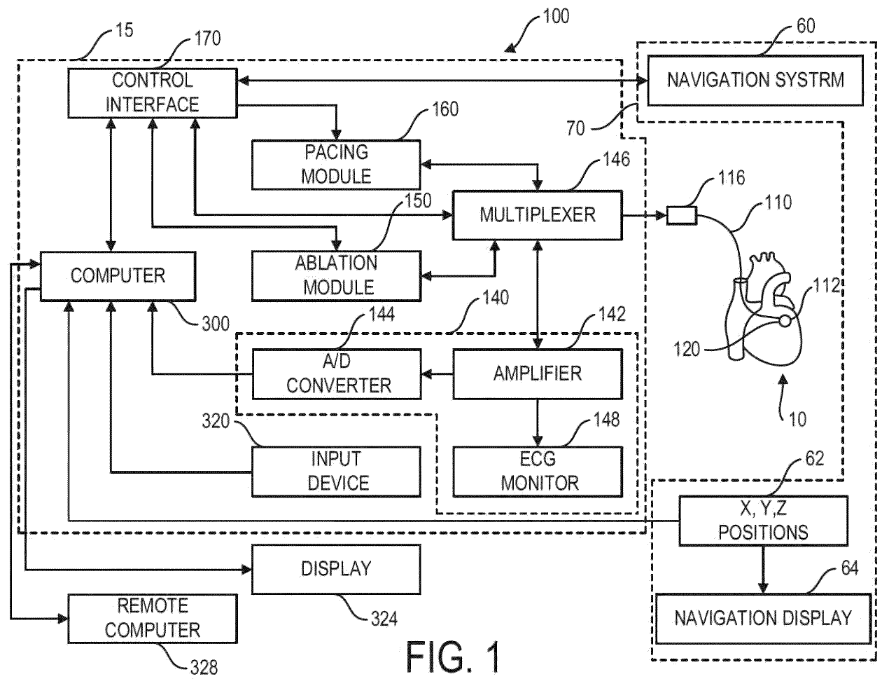

Referring now to FIG. 1, there is illustrated one embodiment of a combined cardiac electrophysiological mapping (EP), pacing and ablation system 100. Note that in some embodiments system 100 may not include ablation module 150, pacing module 160, and/or multiplexer 146. Among other things, the embodiment of system 100 shown in FIG. 1 is configured to detect and reconstruct cardiac activation information acquired from a patient's heart relating to cardiac rhythm disorders and/or irregularities, and is further configured to detect and discover the location of the source of such cardiac rhythm disorders and/or irregularities with enhanced precision relative to prior art techniques. In some embodiments, system 100 is further configured to treat the location of the source of the cardiac rhythm disorder or irregularity, for example by ablating the patient's heart at the detected location.

The embodiment of system 100 shown in FIG. 1 comprises five main functional units: electrophysiological mapping (EP mapping unit) 140 (which is also referred to herein as data acquisition device 140), multiplexer 146, ablation module 150, pacing module 160, imaging and/or navigation system 70, and computer or computing device 300. Data acquisition, processing and control system 15 comprises data acquisition device 140, multiplexer 146, ablation module 150, pacing module 160, control interface 170 and computer or computing device 300. In one embodiment, at least one computer or computing device or system 300 is employed to control the operation of one or more of systems, modules and devices 140, 150, 160, 170 and 70. Alternatively, the respective operations of systems, modules or devices 140, 146, 150, 160, 170 and 70 may be controlled separately by each of such systems, modules and devices, or by some combination of such systems, modules and devices.

Instead of being operably connected (e.g., through Bluetooth signals, a LAN or WAN network, or through the cloud), or directly connected, to computing device 300, data acquisition device 140 may be configured to provide as outputs therefrom saved or stored body surface electrogram signals, which can be, by way of example, saved or stored on a hard drive, in a memory, in the cloud, remotely, on a USB stick, or other suitable storage device, and where the saved or stored body surface electrogram signals are later or subsequently provided as inputs to computing device 300 for processing and analysis.

Computer or computing device 300 may be configured to receive operator inputs from an input device 320 such as a keyboard, mouse and/or control panel, ore remotely. Outputs from computer 300 may be displayed on display or monitor 324 or other output devices (not shown in FIG. 1). Computer 300 may also be operably connected to a remote computer or analytic database or server 328. At least each of components, devices, modules and systems 60, 110, 140, 146, 148, 150, 170, 300, 324 and 328 may be operably connected to other components or devices by wireless (e.g., Bluetooth) or wired means. Data may be transferred between components, devices, modules or systems through hardwiring, by wireless means, or by using portable memory devices such as USB memory sticks.

During electrophysiological (EP) mapping procedures, multi-electrode catheter 110 is typically introduced percutaneously into the patient's heart 10. Catheter 110 is passed through a blood vessel (not shown), such as a femoral vein or the aorta, and thence into an endocardial site such as the atrium or ventricle of the heart 10.

It is contemplated that other catheters, including other types of mapping or EP catheters, lasso catheters, pulmonary vein isolation (PVI) ablation catheters (which can operate in conjunction with sensing lasso catheters), ablation catheters, pulsed field ablation catheters, navigation catheters, and other types of EP mapping catheters such as EP monitoring catheters and spiral catheters may also be introduced into the heart, and that additional surface electrodes may be attached to the skin of the patient to record electrocardiograms (ECGs).

When system 100 is operating in an EP mapping mode, multi-electrode catheter 110 functions as a detector of intra-electrocardiac signals, while optional body surface electrodes may also serve as detectors of surface ECGs. In one embodiment, the analog signals obtained from the intracardiac and/or body surface electrodes may be routed by multiplexer 146 to data acquisition device 140, which comprises an amplifier 142 and an A/D converter (ADC) 144. The amplified or conditioned electrogram signals may be displayed by electrocardiogram (ECG) monitor 148. The analog signals are also digitized via ADC 144 and input into computer 300 for data processing, analysis and graphical display.

In one embodiment, catheter 110 is configured to detect cardiac activation information in the patient's heart 10, and to transmit the detected cardiac activation information to data acquisition device 140, either via a wireless or wired connection. In one embodiment that is not intended to be limiting with respect to the number, arrangement, configuration, or types of electrodes, catheter 110 is a basket catheter that includes a plurality of 64 electrodes, probes and/or sensors A1 through H8 arranged in an 8×8 grid that are included in electrode mapping assembly 120, which is configured for insertion into the patient's heart through the patient's blood vessels and/or veins. Other numbers, arrangements, configurations and types of electrodes in catheter 110 are, however, also contemplated. In most of the various embodiments, at least some electrodes, probes and/or sensors included in catheter 110 are configured to detect cardiac activation or electrical signals to generate electrocardiograms or electrogram signals, and/or to detect constant current electrical signals transmitted from body surface electrodes into the patient's heart (more about which is said below). These signals are then relayed by electrical conductors from or near the distal end 112 of catheter 110 to proximal end 116 of catheter 110 to data acquisition device 140.

Note that in some embodiments of system 100, multiplexer 146 is not employed for various reasons, such as sufficient electrical conductors being provided in catheter 110 for all electrode channels, or other hardware design considerations. In other embodiments, multiplexer 146 is incorporated into catheter 110 or into data acquisition device 140. In still further embodiments, multiplexer 146 is optional or not provided at all, and data acquisition device 140, ablation module 150, and/or pacing module 160 are employed separately and/or operate independently from one another. In addition, in some embodiments computing device 300 may be combined or integrated with one or more of data acquisition device 140, ablation module 150, and/or pacing module 160.

In one embodiment, a medical practitioner or health care professional employs catheter 110 as a roving catheter to locate the site of the location of the source of a cardiac rhythm disorder or irregularity in the endocardium quickly and accurately, without the need for open-chest and open-heart surgery. In one embodiment, this is accomplished by using multi-electrode catheter 110 in combination with real-time or near-real-time data processing and interactive display by computer 300, and optionally in combination with imaging and/or navigation system 70. In one embodiment, multi-electrode catheter 110 deploys at least a two-dimensional array of electrodes against a site of the endocardium at a location that is to be mapped, such as through the use of a Biosense Webster® PENTARAY® EP mapping catheter. The intracardiac or electrogram signals detected by the catheter's electrodes provide data sampling of the electrical activity in the local site spanned by the array of electrodes.

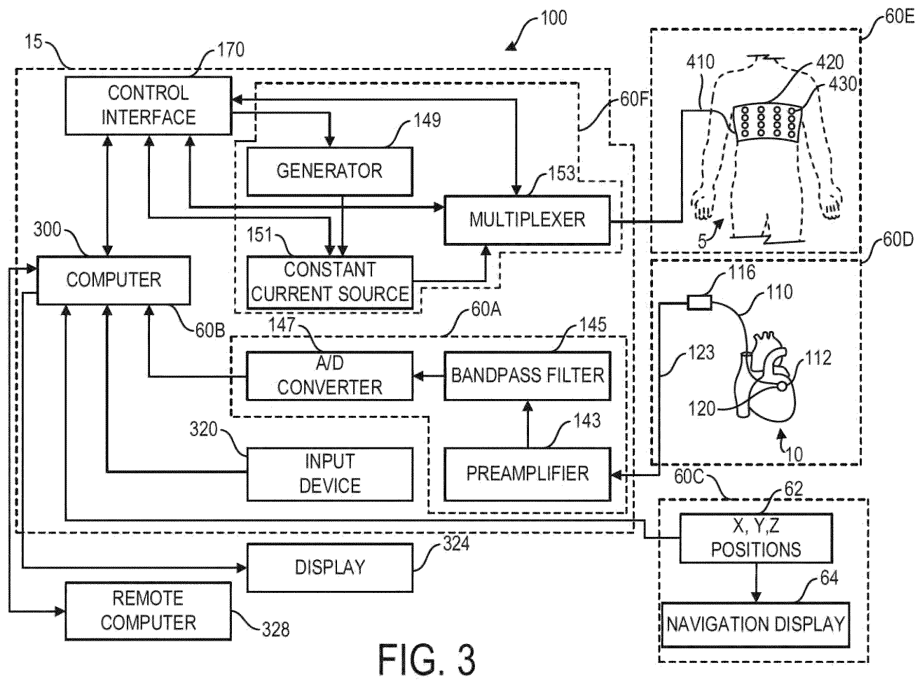

In another embodiment, or in an enhanced or supplemented embodiment, a medical practitioner or health care professional also employs catheter 110 as a roving catheter to locate the site of the location of the source of a cardiac rhythm disorder or irregularity in the endocardium quickly and accurately, where navigation, and positioning of catheter 110 inside the patient's heart is accomplished using the controlled constant current techniques described and disclosed in further detail below (see, for example, FIG. 3).

In one embodiment, the electrogram signal data and/or controlled constant current signal data are processed by computer 300 to produce a display showing the locations(s) of the source(s) of cardiac rhythm disorders and/or irregularities in the patient's heart 10 in real-time or near-real-time, further details of which are provided below. That is, at and between the sampled locations of the patient's endocardium, computer 300 may be configured to compute and display in real-time or near-real-time an estimated, detected and/or determined location(s) of the site(s), source(s) or origin) s) of the cardiac rhythm disorder(s) and/or irregularity(s) within the patient's heart 10, and/or of the electrodes located at or near the distal end of catheter 110. This permits a medical practitioner to move interactively and quickly the electrodes of catheter 110 towards the location of the source of the cardiac rhythm disorder or irregularity.

In some embodiments of system 100, one or more electrodes, sensors or probes detect cardiac activation from the surface of the patient's body as surface ECGs, or remotely without contacting the patient's body (e.g., using magnetocardiograms). In another example, some electrodes, sensors or probes may derive cardiac activation information from echocardiograms. In various embodiments of system 100, external or surface electrodes, sensors and/or probes can be used separately or in different combinations, and further may also be used in combination with intracardiac electrodes, sensors and/or probes inserted within the patient's heart 10. Many different permutations and combinations of the various components of system 100 are contemplated having, for example, reduced, additional or different numbers of electrical sensing and other types of electrodes, sensors and/or transducers.

Continuing to refer to FIG. 1, EP mapping system or data acquisition device 140 may be configured to condition the analog electrogram signals or controlled constant current signals received and delivered by catheter 110 from electrodes A1 through H8 in amplifier 142. Conditioning of the analog electrogram signals received by amplifier 142 may include, but is not limited to, low-pass filtering, high-pass filtering, bandpass filtering, and notch filtering. The conditioned analog signals are then digitized in analog-to-digital converter (ADC) 144. ADC 144 may further include a digital signal processor (DSP) or other type of processor which is configure to further process the digitized electrogram signals (e.g., low-pass filter, high-pass filter, bandpass filter, notch filter, automatic gain control, amplitude adjustment or normalization, artifact removal, etc.) before they are transferred to computer or computing device 300 for further processing and analysis.

As discussed above, in some embodiments, multiplexer 146 is separate from catheter 110 and data acquisition device 140, and in other embodiments multiplexer 146 is combined in catheter 110 or data acquisition device 140.

In some embodiments, the rate at which individual electrogram and/or ECG signals are sampled and acquired by system 100 can range between about 0.25 milliseconds and about 8 milliseconds, and may be about 0.5 milliseconds, about 1 millisecond, about 2 milliseconds or about 4 milliseconds. Other sample rates are also contemplated. While in some embodiments system 100 is configured to provide unipolar signals, in other embodiments system 100 is configured to provide bipolar signals.

In one embodiment, system 100 can include a BARD® LABSYSTEM™ PRO EP Recording System, which is a computer and software driven data acquisition and analysis tool designed to facilitate the gathering, display, analysis, pacing, mapping, and storage of intracardiac EP data. Also in one embodiment, data acquisition device 140 can include a BARD® CLEARSIGN™ amplifier, which is configured to amplify and condition electrocardiographic signals of biologic origin and pressure transducer input, and transmit such information to a host computer (e.g., computer 300 or another computer).

As shown in FIG. 1, and as described above, in some embodiments system 100 includes ablation module 150, which may be configured to deliver RF ablation energy through catheter 110 and corresponding ablation electrodes disposed near distal end 112 thereof, and/or to deliver RF ablation energy through a different catheter (not shown in FIG. 1B). Suitable ablation systems and devices include, but are not limited to, cryogenic ablation devices and/or systems, radiofrequency ablation devices and/or systems, ultrasound ablation devices and/or systems, high-intensity focused ultrasound (HIFU) devices and/or systems, chemical ablation devices and/or systems, and laser ablation devices and/or systems.

When system 100 is operating in an optional ablation mode, multi-electrode catheter 110 fitted with ablation electrodes, or a separate ablation catheter, is energized by ablation module 150 under the control of computer 300, control interface 170, and/or another control device or module. For example, an operator may issue a command to ablation module 150 through input device 320 to computer 300. In one embodiment, computer 300 or another device controls ablation module 150 through control interface 170. Control of ablation module 150 can initiate the delivery of a programmed series of electrical energy pulses to the endocardium via catheter 110 (or a separate ablation catheter, not shown in FIG. 1B). One embodiment of an ablation method and device is disclosed in U.S. Pat. No. 5,383,917 to Desai et al.

In an alternative embodiment, ablation module 150 is not controlled by computer 300, and is operated manually directly under operator control. Similarly, pacing module 160 may also be operated manually directly under operator control. The connections of the various components of system 100 to catheter 110, to auxiliary catheters, or to surface electrodes may also be switched manually or using multiplexer 146 or another device or module.

When system 100 is operating in an optional pacing mode, multi-electrode catheter 110 is energized by pacing module 160 operating under the control of computer 300 or another control device or module. For example, an operator may issue a command through input device 320 such that computer 300 controls pacing module 160 through control interface 170, and multiplexer 146 initiates the delivery of a programmed series of electrical simulating pulses to the endocardium via the catheter 110 or another auxiliary catheter (not shown in FIG. 1B). One embodiment of a pacing module is disclosed in M. E. Josephson et al., in “VENTRICULAR ENDOCARDIAL PACING II, The Role of Pace Mapping to Localize Origin of Ventricular Tachycardia,” The American Journal of Cardiology, vol. 50, November 1982.

In one embodiment, computing device or computer 300 may be appropriately configured and programmed to receive or access the electrogram signals provided by data acquisition device 140. Computer 300 is further configured to analyze or process such electrogram signals in accordance with the methods, functions and logic disclosed and described herein so as to permit reconstruction of cardiac activation information from the electrogram signals. This, in turn, makes it possible to locate with at least some reasonable degree of precision the location of the source of a heart rhythm disorder or irregularity. Once such a location has been discovered, the source may be eliminated or treated by means that include, but are not limited to, cardiac ablation.

In one embodiment, and as shown in FIG. 1, system 100 also comprises a physical imaging and/or navigation or positioning system 70, which may or may not employ the controlled constant current mapping, imaging, navigation, and/or positioning techniques described below. Physical imaging and/or navigation device 60 included in system 70 may be, by way of example, a 2- or 3-axis fluoroscope system, an ultrasonic system, a magnetic resonance imaging (MRI) system, a computed tomography (CT) imaging system, an electrical impedance tomography EIT) system, a controlled constant current system (as described below), or any one or more of the CARTO, Ensite NavX, Rythmia, Affera, Acutus, NavX and CardioNXT systems. Operation of system 70 be controlled by computer 300 via control interface 170, or by other control means incorporated into or operably connected to imaging or navigation or positioning system 70. In one embodiment, computer 300 or another computer triggers physical imaging or navigation system 60 to take “snap-shot” pictures of the heart 10 of a patient (body not shown). A picture image is detected by a detector 62 along each axis of imaging, and can include a silhouette of the heart as well as a display of the inserted catheter 110 and its electrodes A1-H8 (more about which is said below), which is displayed on imaging or navigation display 64. Digitized image or navigation data may be provided to computer 300 for processing and integration into computer graphics that are subsequently displayed on monitor or display 64 and/or 324.

In one embodiment, system 100 further comprises or operates in conjunction with catheter or electrode position transmitting and/or receiving coils or antennas located at or near the distal end of an EP mapping catheter 110, or that of an ablation or navigation catheter 110, which are configured to transmit electromagnetic signals for intra-body navigational and positional purposes.

In one embodiment, imaging or navigation system 60 is used to help identify and determine the precise two- or three-dimensional positions of the various electrodes included in catheter 110 within patient's heart 10, and is configured to provide electrode position data to computer 300. Electrodes, position markers, and/or radio-opaque markers can be located on various potions of catheter 110, mapping electrode assembly 120 and/or distal end 112, or can be configured to act as fiducial markers for imaging or navigation system 70. Alternatively, and as further described below, controlled constant current signals received by sensing or receiving electrodes included in mapping electrode assembly 120 or otherwise located on catheter 110 may be used to navigate or position catheter 110, or to provide images of the locations of such electrodes or portions of catheter 110.

Medical navigation systems suitable or adaptable for use in conjunction with the various embodiments described and disclosed herein include, but are not limited to, image-based navigation systems, model-based navigation systems, optical navigation systems, electromagnetic navigation systems (e.g., BIOSENSE® WEBSTER® CARTO® system), impedance-based navigation systems (e.g., the St. Jude® ENSITE™ VELOCITY™ cardiac mapping system), systems that combine attributes from different types of imaging and navigation systems and devices to provide navigation within the human body (e.g., the MEDTRONIC® STEALTHSTATION® system), and various embodiments of the controlled constant current navigation, positioning and/or imaging system described in detail below.

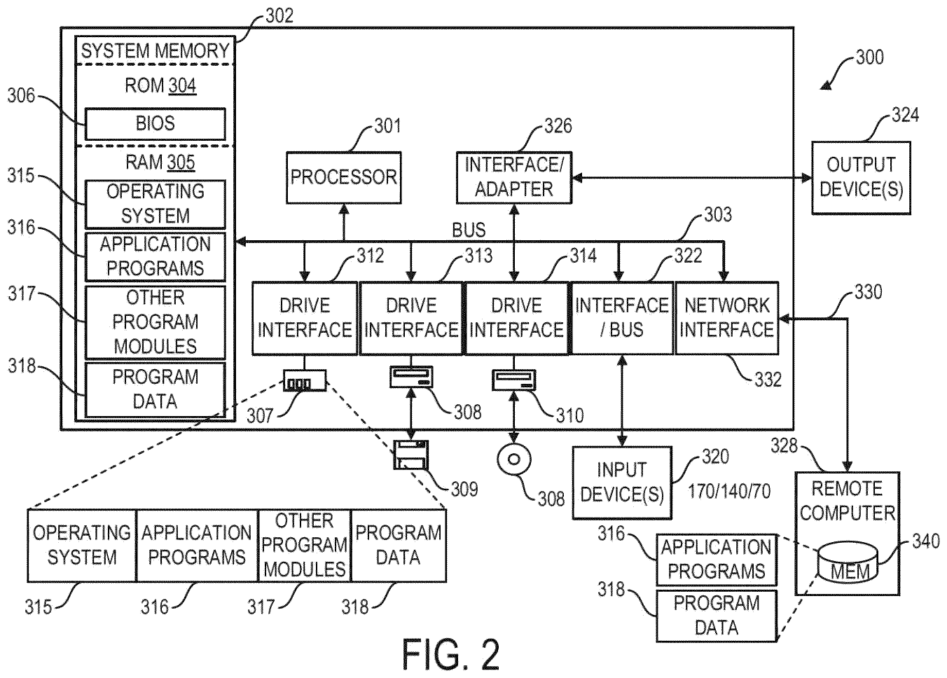

In view of the structural and functional descriptions provided herein, those skilled in the art will appreciate that portions of the described devices and methods may be configured as processes, methods, data processing systems, and/or computer methods. Accordingly, these portions of the devices and methods described herein may take the form of a hardware embodiment, a software embodiment, or an embodiment combining software and hardware, such as shown and described with respect to computer system 300 illustrated in FIG. 2.

Furthermore, portions of the devices and methods described herein may be a process or method stored in a computer-usable storage medium having computer readable program code on the medium. Any suitable computer-readable medium may be utilized including, but not limited to, static and dynamic storage devices, hard disks, optical storage devices, and magnetic storage devices.

Certain embodiments of portions of the devices and methods described herein are also described with reference to block diagrams of methods, processes, and systems. It will be understood that such block diagrams, and combinations of blocks diagrams in the Figures, can be implemented using computer-executable instructions. These computer-executable instructions may be provided to one or more processors of a general purpose computer, a special purpose computer, or any other suitable programmable data processing apparatus (or a combination of devices and circuits) to produce a machine, such that the instructions, which executed via the processor(s), implement the functions specified in the block or blocks of the block diagrams.

These computer-executable instructions may also be stored in a computer-readable memory that can direct computer 300 or other programmable data processing apparatus to function in a particular manner, such that the instructions stored in the computer-readable memory result in an article of manufacture including instructions which implement the function specified in an individual block, plurality of blocks, or block diagram. The computer program instructions may also be loaded onto computer 300 or other programmable data processing apparatus to cause a series of operational steps to be performed on the computer or other programmable apparatus to produce a computer implemented process such that the instructions which execute on computer 300 or other programmable apparatus provide steps for implementing the functions specified in an individual block, plurality of blocks, or block diagram.

In this regard, FIG. 2 illustrates only one example of a computer system 300 (which, by way of example, can include multiple computers or computer workstations) that can be employed to execute one or more embodiments of the devices and methods described and disclosed herein, such as devices and methods configured to acquire and process sensor or electrode data, to process image data, to process received controlled constant current constant electrical signals, and/or transform sensor or electrode data and image data associated with the analysis of cardiac electrical activity and the carrying out of the combined electrophysiological mapping and analysis of the patient's heart 10 and ablation therapy delivered thereto.

Computer system 300 can be implemented on one or more general purpose computer systems or networked computer systems, embedded computer systems, routers, switches, server devices, client devices, various intermediate devices/nodes or standalone computer systems. Additionally, computer system 300 or portions thereof may be implemented on various mobile devices such as, for example, a personal digital assistant (PDA), a laptop computer and the like, provided the mobile device includes sufficient processing capabilities to perform the required functionality.

In one embodiment, computer system 300 includes processing unit 301 (which may comprise a CPU, controller, microcontroller, processor, microprocessor or any other suitable processing device), system memory 302, and system bus 303 that operably connects various system components, including the system memory, to processing unit 301. Multiple processors and other multi-processor architectures also can be used to form processing unit 301. System bus 303 can comprise any of several types of suitable bus architectures, including a memory bus or memory controller, a peripheral bus, or a local bus. System memory 302 can include read only memory (ROM) 304 and random access memory (RAM) 305. A basic input/output system (BIOS) 306 can be stored in ROM 304 and contain basic routines configured to transfer information and/or data among the various elements within computer system 300.

Computer system 300 can include a hard disk drive 303, a magnetic disk drive 308 (e.g., to read from or write to removable disk 309), or an optical disk drive 310 (e.g., for reading CD-ROM disk 311 or to read from or write to other optical media). Hard disk drive 303, magnetic disk drive 308, and optical disk drive 310 are connected to system bus 303 by a hard disk drive interface 312, a magnetic disk drive interface 313, and an optical drive interface 314, respectively. The drives and their associated computer-readable media are configured to provide nonvolatile storage of data, data structures, and computer-executable instructions for computer system 300. Although the description of computer-readable media above refers to a hard disk, a removable magnetic disk and a CD, other types of media that are readable by a computer, such as magnetic cassettes, flash memory cards, digital video disks and the like, in a variety of forms, may also be used in the operating environment; further, any such media may contain computer-executable instructions for implementing one or more parts of the devices and methods described and disclosed herein.

A number of program modules may be stored in drives and RAM 303, including operating system 315, one or more application programs 316, other program modules 313, and program data 318. The application programs and program data can include functions and methods programmed to acquire, process and display electrical data from one or more sensors, such as shown and described herein. The application programs and program data can include functions and methods programmed and configured to process data acquired from a patient for assessing heart function and/or for determining parameters for delivering a therapy and/or assessing heart function, such as shown and described herein.

A health care provider or other user may enter commands and information into computer system 300 through one or more input devices 320, such as a pointing device (e.g., a mouse, a touch screen, etc.), a keyboard, a microphone, a joystick, a game pad, a scanner, and the like. For example, the user can employ input device 320 to edit or modify the data being input into a data processing method (e.g., only data corresponding to certain time intervals). These and other input devices 320 may be connected to processing unit 301 through a corresponding input device interface or port 322 that is operably coupled to the system bus, but may be connected by other interfaces or ports, such as a parallel port, a serial port, or a universal serial bus (USB). One or more output devices 324 (e.g., display, a monitor, a printer, a projector, or other type of display device) may also be operably connected to system bus 303 via interface 326, such as through a video adapter.

Computer system 300 may operate in a networked environment employing logical connections to one or more remote computers, such as remote computer 328. Remote computer 328 may be a workstation, a computer system, a router, or a network node, and may include connections to many or all the elements described relative to computer system 300. The logical connections, schematically indicated at 330, can include a local area network (LAN) and/or a wide area network (WAN).

When used in a LAN networking environment, computer system 300 can be connected to a local network through a network interface or adapter 332. When used in a WAN networking environment, computer system 300 may include a modem, or may be connected to a communications server on the LAN. The modem, which may be internal or external, can be connected to system bus 303 via an appropriate port interface. In a networked environment, application programs 316 or program data 318 depicted relative to computer system 300, or portions thereof, may be stored in a remote memory storage device 340.

Referring now to FIG. 3, there are shown and illustrated various aspects of one embodiment of a controlled constant current navigation or positioning, and optionally imaging, system 60, which in the illustrated embodiment comprises principal components: (a) received constant current signal conditioning and conversion module 60A; (b) computer 60B/300; (c) navigation, position and/or image display module 60C; (d) constant current signal receiving catheter 60D; (e) body surface electrode system 60E, and (f) controlled constant current system 60F. Note that one or more of the various systems, modules, components and devices illustrated in FIG. 3 may be combined with, added to, or operate in conjunction with those illustrated in FIGS. 1 and 2.

In FIG. 3, there is shown a patient 5 wearing a body surface electrode vest 420 comprising a plurality of body surface electrodes 430, which are operably connected to module 60F through electrical connection 410. (Wireless connections may also be employed to replace electrical connection 410.) A high frequency RF generator 149 generates signals that are provided to high-fidelity controlled constant current source 151, which is configured to emit selectable current signals having a degree of accuracy and repeatability. The controlled constant current signals output from constant current source 151 are next provided to multiplexer 153, and thence to medical device/catheter connection 410 and body surface electrodes 430.

Note that electrode vest 420 may be replaced by one or a plurality of electrode strips comprising electrodes 430, by individual electrodes 430 attached or coupled directly to patient 5's body or torso, or any combination thereof. Body surface electrodes 430 are positioned on patient 5's body such that they overlie a volume of interest in patient 5, which in the embodiment illustrated in FIG. 3 includes the patient's heart 10.

Body surface electrodes 430 and module 60F of system 100 are configured to deliver individual, high-fidelity, controlled, constant current, electrical signals to the volume of interest located beneath electrodes 430. In one embodiment, each electrode 430 transmits into the volume or region of interest a controlled constant current signal which is different in phase, amplitude and/or frequency from the controlled constant current signals transmitted by other electrodes 430.

In another embodiment, electrodes 430 may be configured to emit controlled constant current electrical signals that have the same or similar phases, amplitudes and/or frequencies, but which owing to their different locations on the surface of the patient's body, and the different portions of body 5 such signals traverse on their way to sensing or receiving electrodes located within the volume, can result in such signals having different distinguishable characteristics (including, but not limited to, variations in amplitude or phase) when they arrive at the various sensing or reception electrodes located within the volume and patient's body 5.

In yet another embodiment, instead of a “scan” of controlled constant current signals of the same or similar waveform characteristics being transmitted across and through body surface electrodes 430, each body surface electrode 430 transmits a controlled constant current signal having its own unique AC frequency. Such unique AC frequency signals can then be detected by electrodes located on medical device or catheter 110, and using appropriate analog signal bandpass or other digital filtering techniques be differentiated from one another to yield 3D electrode positions.

Thus, and in such embodiments, each body surface electrode 430 transmits a controlled constant current signal that is received by each sensing electrode within the volume as a unique controlled constant current signal owing to, for example, phase, frequency and/or amplitude differences. Together, and with further reference to FIGS. 10 and 14, it will be seen that at each receiving or sensing electrode of medical device or catheter 110 located within the volume, a collective pattern of voltages received by the receiving or sensing electrodes results. In conjunction with one another, the controlled constant current signals transmitted by electrodes 430 and received by the sensing or receiving electrodes of medical device or catheter 110 together permit the X, Y, Z or 3D locations or positions of electrodes located on catheter or medical device 110 and within the volume to be determined, more about which is said below.

Module 60A of system 100 is configured to receive and process the controlled constant current electrical signals transmitted by electrodes 430 and sensed or received by electrodes located on medical device or catheter 110. Such received or sensed controlled constant current electrical signals are routed from medical device or catheter 110 through electrical connection 123 to module 60A. (Wireless connections may also be employed to replace electrical connection 123.) Preamplifier 143 is amplify configured to receive the controlled constant current signals sensed or received thereby, and to condition and convert such signals for further processing by bandpass filter 145. In some embodiments, electrical connection or cable 123 or 410 can be replaced by a wireless connections, such as BLUETOOTH® connection. In one embodiment, and as shown in FIG. 3, constant current signals received by sensing electrodes located on catheter or medical device 110 are directed first to preamplifier 143, then to bandpass filter 145, and last to A/D converter 147 before being received by computer 300 for further processing and analysis. Note that sensing or receiving electrodes located on medical device 110 may also be configured to perform other functions, such as to act as fiducial markers, ablation electrodes, pacing electrodes, defibrillation electrodes, and so on.

In FIG. 3, and in one embodiment, 16 body surface electrodes 430 are mounted on, attached to, or operably coupled to patient 5's thorax or body above the volume (or region of interest). In one embodiment, a corresponding ground electrode pad or one or more ground electrodes are optimally placed diametrically opposite transmitting electrodes 430 on the patient back. Other numbers and configurations of body surface and ground electrodes are also contemplated. As regards ground electrodes, many options exist, including no explicitly provided ground electrode (a ground will nearly always be found for a transmitted electrical signal), substitutes for specific or discrete ground electrodes such as a metal or electrically conductive bed or surgery platforms, pans, leashes, collars, and so on.

As regards transmitting electrodes, the number of such electrodes employed may range, by way of non-limiting example, between 1 electrode and 3 electrodes, 4 electrodes, 8 electrodes, 12 electrodes, 16 electrodes, 24 electrodes, 36 electrodes, 48 electrodes, 64 electrodes, 72 electrodes, 96 electrodes, 128 electrodes, 256 electrodes, 512 electrodes, and 1,024 electrodes, and so on. Some examples of current manufacturers of cardiac monitoring patches, which in at least some cases may be adapted or configured for use as electrode patches configured to transmit or deliver controlled constant current signals, include: (a) iRhythm® and their Zio XT® and Zio TR Patch product offerings; or (b) the Bardy Dx® Carnation Ambulatory Monitor (CAM™). Electrodes configured to operate wirelessly, such as those found in the NUVANT® Mobile Cardiac Telemetry (MCT) Monitor, which communicates wirelessly with a cellular device, are also contemplated. See, for example: (1) U.S. Pat. No. 10,123,703 entitled “Health monitoring apparatus with wireless capabilities for initiating a patient treatment with the aid of a digital computer” to Bardy et al. (“the '703 patent”); (2) U.S. Pat. No. 10,299,691 entitled “Wearable monitor with arrhythmia burden evaluation” to Hughes et al. (“the '691 patent”); (3) U.S. Pat. No. 10,772,522 entitled “Disposable biometric patch device” to Zadig, and (4) “Cardiac Ambulatory Monitoring: New Wireless Device Validated Against Conventional Holter Monitoring in a Case Series” to Murali et al., Front. Cardiovasc. Med., 30 Nov. 2020 (https://doi.org/10.3389/fcvm.2020.587945) describing the SmartCardia® wearable cardiac monitoring patch (“the Murali paper”). Those skilled in the art will realize that certain aspects and features disclosed and described in in the '703 patent, the '691 patent, the '522 patent, and the Murali paper can be employed in, or adapted and modified for use in, the systems, devices, components, and methods described and disclosed herein. The '703 patent, the '691 patent, the '522 patent, and the Murali paper incorporated by reference herein, each in its respective entirety. Apple iWatch®, FitBit®, Galaxy Watch3®, and Galaxy Watch Active2® are examples of watch or watch-like devices configured to acquire cardiac data from the wearer, such as ECGs, blood pressure, heart rate, etc. Such wearable devices likewise contain certain aspects and features that can be employed in, or adapted and modified for use in, the systems, devices, components, and methods described and disclosed herein.

In the example of FIG. 3, there are shown 16 body surface electrodes 430 mounted on the anterior portion of vest 420, which in turn is worn on or attached to the thorax of patient 5. In some embodiments, by way of non-limiting example, another 16 body surface electrodes 430 may be mounted on a posterior surface of vest 420 (not shown in FIG. 3).

It is further contemplated that body surface electrodes 430 may be mounted, attached or coupled to the patient's thorax by means other than a vest, such as by patches, electrode strips, individually, or by other means known in the art. For example, electrode strips manufactured by Goltec GmbH of Cremlingen, Germany can be used. Carbon and metal body surface electrode strips are available from Goltec GmbH. Carbon electrode strips have the advantage of being radio-translucent, i.e., being transparent or substantially transparent during X-ray imaging.

Electrodes may be provided only on the anterior portion of the patient's thorax, only on the posterior portion of the patient's thorax, on side or lateral portions of the patient's thorax, or on any suitable combination of anterior, posterior and/or lateral portions of the patient's thorax.

In applications where the region of interest or volume is not patient 5's heart, transmitting electrodes 430 can be positioned above or below the volume, where the region of interest includes, by way of non-limiting example, patient 5's brain, stomach, kidneys, bladder, colon, large intestine, small intestine, and/or any other internal organ, passageway, or the like which is to be investigated and analyzed using, for example, a catheter or other device configured to be placed inside the human body.

Continuing to refer to FIG. 3, and as mentioned above, in one embodiment electrodes 430 are configured to transmit controlled constant current electrical signals in the direction of or into a volume containing or encompassing patient's heart 10. In addition to transmitting electrodes 430, other types of devices and/or transducers, such as ground electrodes, navigation patches, position markers, or other devices may be configured to operate in conjunction with, be incorporated into, or form a portion of vest 420, electrodes 430, and/or system 100. Electrodes 430 may be reusable or disposable, unipolar or bipolar, and may be configured for use with MRT/MRI, X-Ray, and/or CAT scanning imaging systems or other types of imaging systems 60. Imaging and/or navigation system 60 may also be employed used to help identify and determine the precise positions of the various electrodes 430 or position markers included in vest 430. Gels, adhesives, and liquids may be employed to improve electrical coupling of electrodes 430 with the patient's body, as is well known in the art.

In addition to sensing electrodes 430, other types of devices and/or transducers, such as ground electrodes, navigation patches, position markers, other devices may be configured to operate in conjunction with, be incorporated into, or form a portion of vest 420, electrodes 430, and/or system 10. Electrodes 430 may be reusable or disposable, unipolar or bipolar, and may be configured for use with MRT/MRI, X-Ray, and/or CAT scanning imaging systems or other types of imaging systems 60.

Note that in some embodiments, and as described in some detail above, system 100 of FIGS. 1 and 3 may not include multiplexer 146, ablation module 150, pacing module 160, imaging and/or navigation system 60, or other modules or components shown in FIGS. 1 and 3. Among other things, the embodiments of system 100 shown in FIGS. 1 and 3 may be configured to detect and reconstruct cardiac activation information acquired from a patient's heart relating to cardiac rhythm disorders and/or irregularities, and may further be configured to detect and discover the locations of the source of such cardiac rhythm disorders and/or irregularities with enhanced precision relative to prior art techniques using body surface electrodes 430. In some embodiments, system 100 is further configured to treat the location of the source of the cardiac rhythm disorder or irregularity, for example by ablating the patient's heart at the detected source location.

The embodiment of system 100 shown in FIGS. 1 and 3 may include at least one computer or computing device or system 300 employed to control the operation of one or more of systems, modules and/or devices included in 60, 70, 100, 140, 150, 160, and 170. Alternatively, the respective operations of systems, modules or devices 60, 70, 100, 140, 150, 160, and 170 may be controlled separately by each of such systems, modules and devices, or by some combination of such systems, modules and devices.

Computer or computing device 300 may be configured to receive operator inputs from an input device 320 such as a keyboard, mouse and/or control panel. Outputs from computer 300 may be displayed on display or monitor 324 or other output devices (not shown in FIGS. 1 and 3). Computer 300 may also be operably connected to a remote computer or analytic database or server 328. At least each of components, devices, modules and systems 60, 70, 100, 110, 140, 146, 148, 150, 170, 300, 324, 328, 410, 420, and 430 may be operably connected to other components or devices by wireless (e.g., BLUETOOTH) or wired means. Data may be transferred between components, devices, modules or systems through hardwiring, by wireless means, or by using portable memory devices such as USB memory sticks.

During navigation, positioning, imaging, body surface EP mapping, and/or EGF analysis procedures, and as described above, body surface electrodes 430 are positioned on the thorax of patient 5, and by way of example may be mounted on a vest 420 that is configured to place individual electrodes 430 in predetermined positions on the patient's body. These predetermined electrode positions can also be provided to imaging and/or positioning or navigation system 60 and/or to computer 300 as a data file so that the spatial positions of body surface electrodes 430 are known (at least approximately), and so that EGF and/or navigation/positioning/imaging analysis can be carried out as described and disclosed herein.

When system 100 of FIGS. 1 and 3 is operating in an EP mapping or EGF mode, body surface electrodes 430, or other body surface electrodes, may also function as detectors of electrocardiographic signals. In one embodiment, the analog signals obtained from body surface electrodes 430 can be routed by multiplexer 146 to data acquisition device 140, which comprises an amplifier 142 and an A/D converter (ADC) 144. The amplified or conditioned electrogram signals may be displayed by electrocardiogram (ECG) monitor 148. The analog signals may also be digitized via ADC 144 and input into computer 300 for data processing, EGF analysis and graphical display.

In one embodiment, controlled constant current medical device navigation, positioning and imaging technology employs an approximately two-dimensional scanning method, where a controlled constant current source is located over the chest of patient 5 where heart 10 is located. As described above, and in one embodiment, scanning is performed using a matrix or array of skin or body surface electrodes 430 driven by a multiplexer 153, and applying a spherically divergent field of electrical current in the long wave RF range of frequency. By reading the induced voltage pattern at locations within the volume or matrix using catheter receiving or sensing electrodes, and reconstructing a 3D matrix from the induced voltage pattern, the exact positions of the catheter receiving electrodes within the body and the volume can be determined with a great deal of locational precision.

By applying the current generated by controlled constant current sources located at the patient's body surface, the voltage profile created by the current within the heart chambers and the volume is independent from the access resistances of individual electrodes 430. The matrix of electrodes 430 of known spacing D1 and D2 allows the controlled constant current scan to directly provide absolute rectangular coordinates.

To compensate for movements of the atria with heartbeat, breathing, and patient movements, in one embodiment a coronary sinus (CS) or other catheter can be used as a reference. As a second reference system, the local shape of a QRS complex in an electrogram, which differs depending on a catheter electrode's location, can be mapped to 3D space using, for example, a neural network where the QRS shapes detected at each CS electrode on a CS catheter are monitored in real time or near-real-time, and shifts in position of the catheter are detected by determining whether the shapes of the QRS complexes exceed a predetermined threshold or other pertinent parameter. See: (a) U.S. patent application Ser. No. 17/831,249 to Tenbrink et al. entitled “Methods, Systems, Devices, and Components for Extracting Atrial Signals from QRS and QRST Complexes” filed on Jun. 2, 2022 (hereafter “the '249 patent application); (b) U.S. patent application Ser. No. 17/863,246 to Denner et al. entitled “Biosignal-Based Intracardiac Navigation Systems, Devices, Components and Methods” filed on Jul. 12, 2022 (hereafter “the '246 patent application); and (c) U.S. patent application Ser. No. 18/125,630 to Grund et al. entitled “Systems, Devices, Components and Methods for Electroanatomical Mapping of the Heart Using 3D Reconstructions Derived from Biosignals” filed on Mar. 23, 2023 (hereafter “the '630 patent application). The '249, '246, and '630 patent applications are incorporated by reference herein, each in its respective entirety.

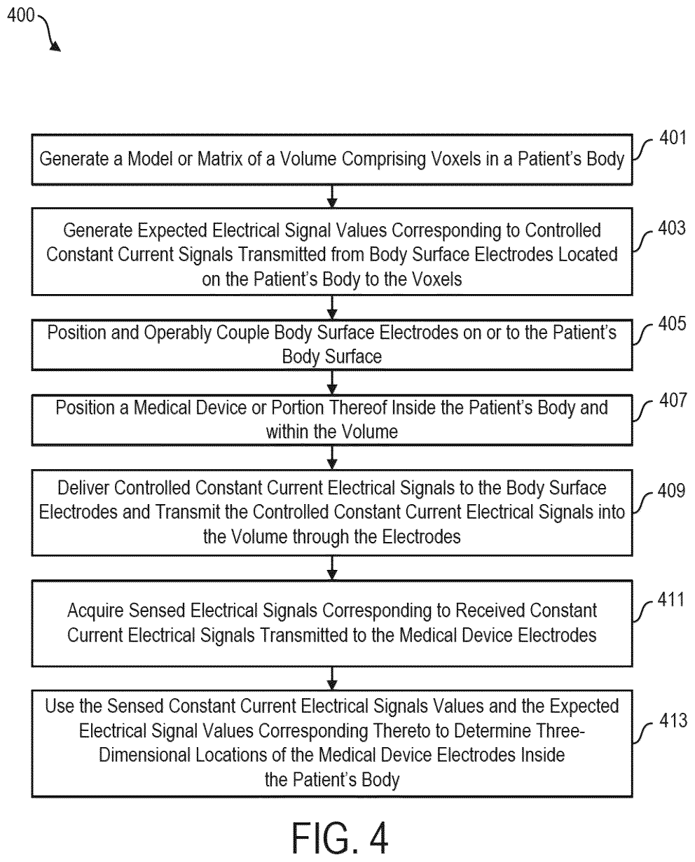

Referring now to FIGS. 4-23, overall controlled constant current technology will be seen to offer a unique approach to navigation, positioning and imaging that provides accurate, high-resolution imaging of the heart or a medical device without being affected by electrode access or input resistances or impedances, or patient movements.

FIG. 4 illustrates one method of employing some of the embodiments of the controlled constant current navigation, positioning and/or imaging systems described and disclosed herein. In method 400 of FIG. 4, at step 401 a model of a volume, or region or volume of interest, comprising voxels is generated for the patient's body. In some embodiments, the model or volume is specifically generated to include and focus upon patient's heart 10, and may incorporate details regarding a specific patient's age, size, sex, body mass index (BMI), fatness, leanness, or thickness of tissue, musculature, bone characteristics, and/or cardiac characteristics (e.g., cardiomyopathy, etc.). At step 402, electrical signal values corresponding to controlled constant current signals transmitted from body surface electrodes 430 located on the patient's body 5 to the voxels in the volume are generated. At step 405, body surface electrodes 430 are positioned and operably coupled on or to patient's 5's body surface. At step 407, a medical device or portion thereof (such as a catheter) is positioned inside the patient's body and within the volume, where the medical device comprises receiving or sensing electrodes configured to receive controlled constant current signals transmitted by body surface electrodes 430. At step 409, controlled constant current electrical signals are delivered to body surface electrodes 430 and transmitted into the volume or region of interest through electrodes 430. At step 411, electrical signals corresponding to constant current electrical signals transmitted to and received by sensing electrodes mounted on near the medical device are acquired by the sensing ort receiving electrodes located on the medical device. At step 413, the sensed constant current electrical signals values and the expected electrical signal values corresponding thereto are employed to determine the three-dimensional locations of the medical device sensing or receiving electrodes located inside the patient 5's body and within the volume or region of interest. The expected electrical signal values are generated using the known predetermined amplitudes, phases, and/or frequencies of the unique transmitted controlled constant current signals generated, or that are to be generated, by constant current source 151 and transmitted into the volume or region of interest by electrodes 430 for reception or sensing by electrodes located on medical device or catheter 110.

In respect of foregoing steps 410 through 413, further aspects of such steps may include, but are not limited to, one or more of the following: (a) body surface electrodes 430 being configured for placement on or over a first portion of the patient 5's body surface; (b) a plurality of receiving or sensing electrodes mounted on or attached to the catheter or medical device 110, each such electrode having a predetermined location or position on or in the catheter or medical device; (c) at least one controlled constant current source 151 configured to be operably connected to the plurality or selected ones of the body surface electrodes 430 and to transmit controlled constant current signals therethrough; (d) a data acquisition or recording device 60A operably connected to at least one computing device 60B/300, the data acquisition or recording device 60A being operably connected to the catheter or medical device electrodes and configured to acquire, or store or record, electrical signals corresponding to transmitted controlled constant current signals sensed by at least some of the plurality of catheter electrodes as sensed electrical signals; (e) the data acquisition or recording device 60A further being configured to relay sensed electrical signals to computing device 60B/300 as sensed electrical signal values, the at least one computing device 60B/300 comprising at least one non-transitory computer readable medium configured to store instructions executable by at least one processor to permit navigation or positioning of the catheter or other type of medical device 110, or a portion thereof, inside the patient 5's body; (e) using the at least one computing device 60B/300, generating at least one three-dimensional model or matrix of a volume of a portion of the patient's body through or into which catheter or medical device 100 (or a portion thereof) is to be navigated or positioned, the volume comprising a plurality of voxels, each voxel having a three-dimensional (3D) spatial coordinate within the volume; (f) using the at least one computing device 60B/300, and for each voxel or selected ones of the voxels, generating expected electrical signal values corresponding to controlled constant current signals transmitted from body surface electrodes 430 to each voxel or selected ones of the voxels; (g) positioning and operably coupling the plurality of body surface electrodes 430 on or to the first portion of the patient's body surface; (h) positioning the catheter or medical device 110, or a portion thereof, inside the patient's body and within at least a portion of the volume; (h) delivering, over a given period of time, the controlled constant current signals to the plurality or selected ones of the body surface electrodes 430 for transmission into at least portions of the volume; (i) using the data acquisition or recording device 60A and the at least one computing device 60B/300, acquiring the sensed electrical signals from the plurality or selected ones of the catheter or medical device electrodes 430 during the given period of time, and storing or recording the sensed electrical signal values corresponding to the sensed electrical signals, the sensed electrical signals corresponding to controlled constant current signals transmitted by the plurality or selected ones of the body surface electrodes 430 into at least portions of the volume during the given period of time; (j) using the at least one computing device 60B/300, and for at least a portion of the given period of time, determining, on the basis of the sensed electrical signal values and the expected electrical signal values corresponding thereto, at least one three-dimensional location of at least one of the catheter or medical device electrodes located within the patient's body and the at least portion of the volume during the given period of time.

Still further aspects of employing some of the embodiments of the controlled constant current navigation, positioning and/or imaging systems described and disclosed herein include, but are not limited to, the following:
following the given period of time, and during subsequent given periods of time, continuing to: (i) transmit controlled constant current signals through the body surface electrodes 430 into patient's body 5 and the at least portion of the volume;
acquire sensed electrical signals; (c) store or record sensed electrical signal values, and (d) determine, on the basis of the sensed electrical signal values and expected electrical signal values corresponding thereto, subsequent three-dimensional locations of the catheter or medical device electrodes located within patient's body 5 and the at least portion of the volume during each or selected ones of the subsequent given periods of time, thereby to permit navigation, positioning and/or imaging of the catheter or medical device 100, or portion thereof, inside patient's body 5.

Yet further aspects of employing some of the embodiments of the controlled constant current navigation, positioning and/or imaging systems described and disclosed herein include, but are not limited to, the following: (a) the at least one three-dimensional location of at least one of the catheter or medical device electrodes is located within patient's heart 10, and further wherein the at least portion of the volume is located within the patient's heart; (b) using controlled constant current signals sensed by or transmitted from a coronary sinus catheter to compensate for at least one of movements of the patient's atria with heartbeat, movements resulting from the patient breathing, and other patient movements; (c) sensing cardiac electrical signals from the patient's heart, determining at least one shape of the patient's QRS complex from the cardiac electrical signals, and verifying or improving the accuracy of the determination of the at least one three-dimensional location provided by the controlled constant current navigation, positioning or imaging system 100; (d) using the sensed electrical signals, reconstructing and displaying a geometry or visual model of the catheter or medical device 110; (e) using the sensed electrical signals, generating an anatomical shell representation of at least a portion of an interior the patient's heart 10 and displaying the anatomical shell representation on display 64; (f) wherein the catheter 110 is one or more of a basket catheter, an electrophysiological mapping catheter, a lasso catheter, a fan-shaped catheter, an umbrella-shaped catheter, a pulsed field ablation (PFA) catheter, a coronary sinus catheter, or an ablation catheter; (g) wherein at least some of the plurality of catheter electrodes comprise one or more of sensing electrodes, ablation electrodes, electrophysiological (EP) mapping electrodes, and navigation electrodes; (h) wherein at least one corresponding ground electrode for use in conjunction with the plurality of body surface electrodes 430 is provided; (i) wherein at least one ground electrode is configured for placement on or over a second portion of the patient's body surface; (j) wherein the plurality of body surface electrodes 430 are configured in an array; (k) wherein the body surface electrode array is configured in at least one of a cross shape, a triangular shape, a strip or linear shape, a plurality of strips or linear shapes, a square shape, a rectangular shape, a star shape, a round shape, an oval shape, an elliptical shape, a geometrically irregular shape, and a geometrically regular shape; (l) wherein the at least one controlled constant current source 151 is at least one of an alternating current (AC) source and a direct current (DC) source; (m) wherein the at least one controlled constant current source 151 is configured to deliver constant current AC signals ranging between about 1 kHz and about 1 MHz in frequency; (n) wherein the at least one controlled constant current source is further configured to generate and deliver constant current AC signals having amplitudes ranging between about 0.1 mA and about 100 mA; (o) wherein the at least one controlled constant current source is further configured to be sequentially connected to the plurality of body surface electrodes 430 and to deliver sequentially controlled constant current signals thereto, for example through multiplexer 153.; (p) wherein the at least one controlled constant current source 151 is further configured to be sequentially connected to each of the plurality of body surface electrodes 430 between about once every 10 milliseconds and about once every 500 milliseconds; (q) wherein the at least one controlled constant current source 151 is further configured to be sequentially connected to each of the plurality of body surface electrodes 430 between about once every 100 milliseconds and once about every 300 milliseconds; (r) wherein the data acquisition or recording device 60A further comprises amplifiers 145 and filters 143 configured to amplify and filter the sensed electrical signals; (s) wherein the amplifiers 145 and filters 143 are configured to at least one of amplify, bandpass filter, notch filter, low-pass filter, high pass filter, and digitally filter the sensed electrical signals; (t) wherein the at least one three-dimensional location of at least one of the catheter or medical device electrodes located within the patient's body 5 and the at least portion of the volume is determined with an accuracy of about 2 mm or less; (u) wherein a number of the plurality of body surface electrodes ranges between about 2 and about 128, or between about 4 and about 64, or between about 8 and 32; (v) wherein a number of the plurality of body surface electrodes 430 ranges between about 4 and about 32; (w) wherein a number of the plurality of catheter electrodes ranges between about 1 and about 256; (x) wherein a number of the plurality of catheter or medical electrodes ranges between about 8 and about 128, or between about 16 and about 64; (y) wherein the at least one three-dimensional model or matrix of the volume is generated according to at least one of the patient's body mass index (BMI), sex, weight, size, and age; (z) wherein the medical device or catheter 110, or portion thereof, is configured to be inserted into a patient's vein or artery and moved therethrough or therein; (aa) wherein the medical device or catheter 110, or portion thereof, is configured to be inserted into one of a patient's coronary artery, brain, throat, esophagus, stomach, liver, urinary tract, colon, orifice, or other body organ, tissue or passageway.

As regards the aforementioned volume or region of interest comprising voxels, voxels are individual volume elements, and each voxel represents a value in a three dimensional space, which in turn can correspond to a pixel for a given slice thickness. Voxels are frequently used in the visualization and analysis of medical data, such as MRIs.

In one embodiment of a controlled constant current navigation, positioning and imaging system, and in respect of a volume or region of interest encompassing heart 10, the volume or region of interest has dimensions of 15 cm×15 cm×15 cm, and individual voxels having dimensions of 1 mm×1 mm×1 mm, for a total of 3,375,000 voxels. During navigation, receiving or sensing electrode 3D positions within the volume are associated with specific voxels within the volume, more about which is said below.

Additional details regarding the structure, operation and functionality of the controlled constant current navigation, positioning and imaging system are described and illustrated in detail in co-pending and commonly-assigned U.S. patent application Ser. No. 18/388,796 made by the inventors of the present disclosure. The disclosure of U.S. patent application Ser. No. 18/388,796 is incorporated herein by reference in its entirety and for all purposes.

In one embodiment, the controlled constant current navigation, positioning and imaging system of the present disclosure includes, as a component thereof, a multi-channel (e.g., 128-channel) Near-Field Differential Complex Impedance (NFDCI) measurement system designed to provide granular impedance and phase measurement data pertinent to each intracardiac catheter electrode described above. As explained in greater detail below, the NFDCI system permits enhanced and improved understanding and monitoring of tissue-electrode contact, inherent tissue attributes, and electrode health and freedom of short circuits by sensing and measuring differential complex impedance. The constant current navigation, positioning and imaging system including the NFDCI system has particular advantages when used in conjunction with PFA ablation techniques, as described in detail below.

As is known, reversible PFA operates by applying electric fields that temporarily increase cell membrane permeability without causing permanent damage. This process involves the creation of nanopores in the cell membrane, disrupting its usual ionic permeability and capacitance. In contrast, irreversible PFA induced by higher intensity electric fields or longer exposure causes permanent cell membrane disruption and cell death, resulting in complete destruction of the cell structure and in some cases the extracellular matrix.

As is further known, alpha and beta dispersion are phenomena observed in the frequency-dependent behavior of tissue conductivity and permittivity. Alpha dispersion typically occurs at lower frequencies, ranging from a few hertz to a few kilohertz, and is primarily influenced by the polarization of cell membranes and the movement of ions at interfaces. As the frequency increases, the capacitive reactance of cell membranes decreases, leading to an increase in conductivity. Beta dispersion, observed at higher frequencies (from a few kilohertz to several megahertz), is associated with the relaxation processes of cell membranes and the Maxwell-Wagner interfacial polarization. This dispersion reflects the properties of both intracellular and extracellular components, providing insight into the dielectric behavior of the tissue at these frequencies. See Schwan, H. P. (1959). Alternating current spectroscopy of biological substances. Proceedings of the IRE, 47(11), 1841-1855.

Pulsed field ablation (PFA), whether reversible or irreversible, impacts the electrical properties of biological tissues, particularly influencing alpha and beta dispersion. These dispersions reflect how the tissue's conductivity and permittivity change with frequency, and understanding these changes is the basis of our invention for optimizing PFA ablation techniques utilized to achieve pulmonary vein isolation (PVI).

Reversible PFA involves the temporary creation of nanopores in the cell membrane, which disrupts the usual ionic permeability and capacitance of the membrane. Such changes shift the alpha dispersion, typically observed at low frequencies (a few Hz to a few kHz), to higher frequencies. This shift is due to the modified capacitive properties of the cell membrane, which now respond differently to the applied electric fields. Consequently, at frequencies below 100 kHz, the increased permeability can lead to decreased conductivity. This reduction occurs because the capacitive effects of the cell membrane diminish, and the overall resistive path of ionic movement becomes more prominent.

In contrast, irreversible PFA results in the complete destruction of the cell membrane and often the extracellular matrix. It is believed that such extensive damage eliminates the structures responsible for beta dispersion, typically occurring at frequencies from a few kHz to a few MHz. Without these structures, the frequency-dependent dielectric properties contributing to beta dispersion are lost. As a result, the tissue may exhibit relatively constant conductivity up to the MHz range. The loss of the heterogeneous components that cause dispersion, like intact cell membranes and intracellular structures, leads to more homogeneous electrical behavior across this frequency range.

The NFDCI system described herein utilizes the foregoing dispersion properties to monitor and ensure the effectiveness of PFA procedures in real-time. NFDCI measures changes in tissue voltage vs current phase shift at a selected frequency (e.g., 100 kHz), providing immediate feedback on the extent of tissue permeabilization and ablation. In this way, the NFDCI system can assist the clinician in optimizing PFA techniques, ensuring targeted and effective ablation while minimizing collateral damage. By tailoring the PFA approach to full irreversibility using NFDCI, PFA procedures may achieve better outcomes and reduce the risks associated with maximized ablation energy.

The fundamentals of the operation of the NFDCI system are as follows: (a) a constant AC current (e.g., 100 kHz) is injected via one or more selected electrodes and a voltage drop is sensed utilizing two different electrodes; (b) complex electrical impedance is calculated to reveal low- and high-frequency current paths through tissue; (c) a corresponding circuit model represents complex tissue impedance comprising extracellular (Re) resistances, intracellular (Ri) resistances, and cell membrane capacitance (Cm); and (d) irreversible electroporation pulse sequences with alternating polarity are applied to ablate the tissue; and impedance frequency plots display changes in capacitive and ohmic resistance as a result of electroporation, highlighting the capacitive resistance change of cell membranes (notably in phase angle) when pulsed electric fields permeabilize cellular membranes.

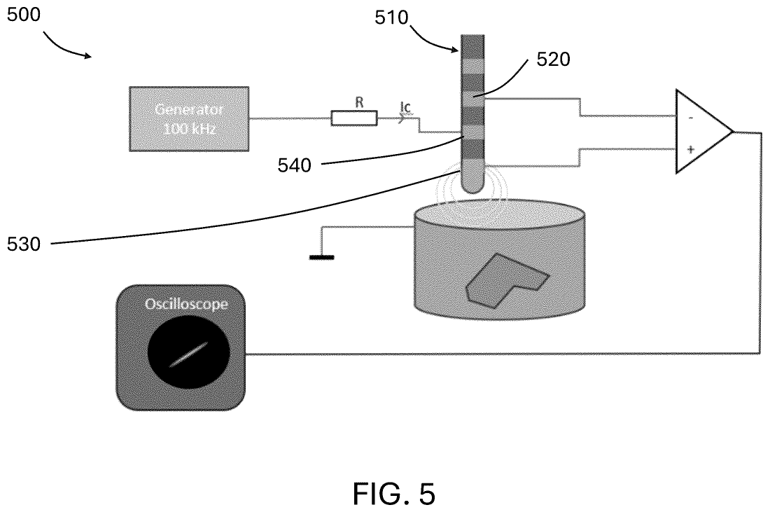

According to one embodiment, a working principle of the multi-channel NFDCI system may be demonstrated using a set of three electrodes located near the tip of a PFA ablation catheter 510 as shown schematically in FIG. 5. By subtracting the responses of two sensing electrodes (electrodes 520 and 530) to an injection of a constant (e.g., 100 kHz) current signal mediated by a third injection electrode (electrode 540), a synchronous change in the amplitude and phase of the detected signal is observed when one of the detecting electrodes is in close proximity to living tissue.

For single-point ablation procedures, and in one embodiment, the system utilizes a three-electrode configuration, as previously explained. This setup includes the ablation electrode (tip), a secondary electrode for current injection, and a third electrode that, along with the tip, is used to record impedance and phase. Many other ablation, current injection, and recording/sensing electrode configurations and numbers of electrodes are contemplated, however, as will be appreciated by those of skill in the art after having read and understood the teachings set forth herein regarding NFDCI. In addition, the NFDCI teachings set forth herein may also be expanded for use in conjunction with, or in addition to, the controlled constant current navigation, positioning and imaging methods, systems, devices and components described herein.

Applying a constant current (e.g., of approximately 1 mA) to the electrode 540 results in a spherical distribution of the current relative to a distant ground electrode, typically placed on the patient's back. The tip electrode 530 senses an electric voltage primarily defined by the voltage drop in the first few millimeters around the tip electrode due to the divergence of the electric current. This voltage drop follows an inverse relationship with distance (1/x), meaning half of the voltage drop occurs within a distance equal to the spacing between the first and second electrodes. Consequently, the effective sensing depth is around 4 mm. Within this region, the capacitive component in the beta dispersion domain dominates the phase of the recorded voltage.

In some embodiments, when dealing with multi-point ablation catheters and diagnostic catheters, an NFDCI system can be configured to employ an alternating current injection strategy across odd and even electrodes. Odd-numbered electrodes (e.g., 1, 3, 5 . . . ) can serve as recording or measurement electrodes in one phase, while even-numbered electrodes (e.g., 2, 4, 6 . . . ) can be configured to emit the current. During this phase, the system captures the difference in impedance and phase between each recording electrode and the averaged response of all recording/measurement electrodes.

In the subsequent phase, the roles may be reversed to ensure a comprehensive assessment of the tissue, with the even-numbered electrodes recording and the odd-numbered electrodes injecting current.

The signals generated by these processes are then relayed to an amplifier and filter unit, which cleans up the signals for better precision. In one embodiment, filtered signals can then be processed by a Phase-Locked Loop (PLL) and an RMS converter. The PLL is instrumental in extracting phase information from the impedance signal, while the RMS converter is used to translate the varying electrical signals into a stable form that reflects the root mean square of the signal, which is particularly useful for identifying the impedance magnitude.

In one embodiment, the entire process can be configured to repeat every 200 milliseconds, enabling real-time monitoring and adjustment during the delivery of ablation therapy. In one embodiment, each 200 milliseconds sequence contains 24 segments; the first 16 segments represent the time intervals during which 10 mA, 100 kHz signals are multiplexed among the 16 body surface electrodes for navigation purposes. Each of these segments is 9 milliseconds long. The following 8 segments are each 5 milliseconds long and represent four pairs of impedance and phase recordings from (1) the odd and even ablation electrodes, and (2) the odd and even diagnostic electrodes, respectively. The sequence concludes with a 16 milliseconds pause to establish a zero-signal baseline.

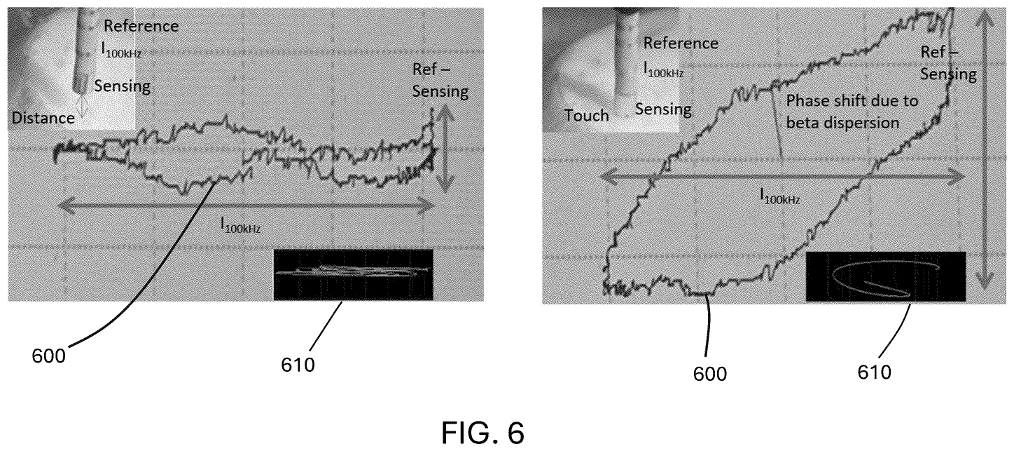

FIG. 6 illustrates the change in the current versus voltage Lissajous figure from flat to round as the point ablator approaches the surface of a potato, indicating beta dispersion-dependent phase shifts in the potato tissue: raw data recorded from a point ablator at a 3 millimeter distance (left) and when touching the surface of a potato (right). With reference to FIG. 6, the line 600 represents the original signal from an oscilloscope, with the horizontal axis showing the injected 100 kHz AC current and the vertical axis showing the voltage difference between the sensing electrode at the tip of the ablator and the third reference electrode. Upon touching the surface, the phase shift at the sensing electrode creates an almost spherical Lissajous figure, reflecting the phase shift induced by beta dispersion at a frequency of 100 KHz. Same result was found when touching atrial myocardium in a swine (inlays 610).

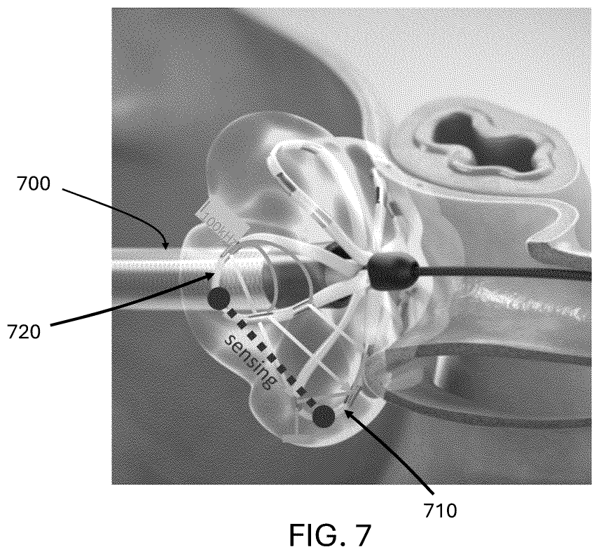

The NFDCI system of the various embodiments may also be useful in a PFA system utilizing a multi-splined catheter. FIG. 7 illustrates a splined PFA catheter 700 having a plurality of splines, each with a plurality of electrodes disposed along its length. The particular embodiment illustrated in FIG. 7 may correspond to the FARAWAVE™ pulsed field ablation catheter manufactured and marketed by Boston Scientific Corporation. In embodiments, the NFDCI system of the present disclosure utilizes electrodes on two splines. In the illustrated example, one or more electrodes on the spline 710 are connected to ground, while one or more electrodes on the adjacent spline 720 receive a constant current AC signal (e.g., a 1 mA, 100 kHz signal). An additional electrode (i.e., other than the current injecting electrode(s)) on the spline 710 and/or 720 are utilized to sense the corresponding voltage as an input to the NFDCI system, which tracks the voltage amplitude and phase shift as explained above.

Because the electrical resistance between the electrodes on the adjacent splines 710, 720 is necessarily lower than the resistance between a spline electrode and an extracardiac indifferent electrode (e.g., a body surface electrode), the majority of the injected current will flow from the spline 720 (the injecting spline) to the spline 710 (the ground spline). The NFDCI system analyzes alpha and beta dispersion as explained above. If there is a capacitive current component of the beta dispersion between the electrodes on the splines 710, 720, this will be visible as a phase shift in the voltage read by sensing electrode(s). As further explained above, a phase shift approaching zero with a concurrent decline in conductance is indicative of complete ablation of the cardiac tissue between the splines 710, 720.

In embodiments, the NFDCI system of the present disclosure can be configured to augment and work in conjunction with the navigation system, aiding in the interpretation of local tissue conditions during ablation or electrophysiological mapping procedures.

Further embodiments of medical navigation, mapping and PFA systems, devices, components and methods will become apparent to those skilled in the art after having read and understood the claims, specification and drawings hereof.

It will now be seen that the various systems, devices, components and methods disclosed and described herein are capable of permitting a medical device to be navigated, positioned and imaged inside a human body quickly, and with considerable accuracy and precision, thereby permitting the delivery of better informed and more accurate and likely-to-succeed treatment decisions for patients. It will also now be seen that the various systems, devices, components and methods disclosed and described herein are capable of more accurate, speedier, more cost-effective, and more efficient ablation of cardiac tissue, thereby permitting the delivery of better informed and more accurate and likely-to-succeed treatment and treatment decisions for patients.

In view of the structural and functional descriptions provided herein, those skilled in the art will appreciate that portions of the described devices and methods may be configured as methods, data processing systems, or computer methods. Accordingly, these portions of the devices and methods described herein may take the form of a hardware embodiment, a software embodiment, or an embodiment combining software and hardware, such as shown and described with respect to computer system 300 illustrated in FIG. 2. Furthermore, portions of the devices and methods described herein may be a computer method stored in a computer-usable storage medium having computer readable program code on the medium. Any suitable computer-readable medium may be utilized including, but not limited to, static and dynamic storage devices, hard disks, optical storage devices, and magnetic storage devices.

Certain embodiments of portions of the devices and methods described herein are also described with reference to block diagrams of methods, systems, and computer methods. It will be understood that such block diagrams, and combinations of blocks diagrams in the Figures, can be implemented using computer-executable instructions. These computer-executable instructions may be provided to one or more processors of a general purpose computer, a special purpose computer, or any other suitable programmable data processing apparatus (or a combination of devices and circuits) to produce a machine, such that the instructions, which executed via the processor(s), implement the functions specified in the block or blocks of the block diagrams.

These computer-executable instructions may also be stored in a computer-readable memory that can direct computer 300 or other programmable data processing apparatus to function in a particular manner, such that the instructions stored in the computer-readable memory result in an article of manufacture including instructions which implement the function specified in an individual block, plurality of blocks, or block diagram. The computer program instructions may also be loaded onto computer 300 or other programmable data processing apparatus to cause a series of operational steps to be performed on the computer or other programmable apparatus to produce a computer implemented process such that the instructions which execute on computer 300 or other programmable apparatus provide steps for implementing the functions specified in an individual block, plurality of blocks, or block diagram.

In this regard, FIG. 2 illustrates only one example of a computer system 300 (which, by way of example, can include multiple computers or computer workstations) that can be employed to execute one or more embodiments of the devices and methods described and disclosed herein, such as devices and methods configured to acquire and process sensor or electrode data, to process image data, and/or transform sensor or electrode data and image data associated with the analysis of cardiac electrical activity and the carrying out of the combined electrophysiological mapping and analysis of the patient's heart 10 and ablation therapy delivered thereto (in addition to navigation, positioning and imaging modalities). Likewise, systems 100 shown in FIGS. 1 and 3 may be modified to permit the acquisition of both body surface and intra-cardiac electrode data simultaneously or sequentially.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An intracardiac pulse field ablation (PFA) system comprising a catheter configured to ablate, and measure near-field complex electrical impedance of, cardiac tissue inside a patient's heart, a data acquisition, recording or measurement device, a constant current AC signal generator, and at least one computing device, wherein the PFA catheter comprises a proximal end and a distal end, a catheter body located between the proximal end and the distal end, and a plurality of electrodes including at least first, second and third electrodes, the PFA catheter and the electrodes thereof being configured to be operably connected to the data acquisition, recording or measurement device, the constant current AC signal generator, and at least one computing device, the first, second, third and fourth electrodes being located near or in the direction of the distal end, the first electrode being operable as a signal injection electrode configured to inject constant current AC signals into or near the patient's cardiac tissue, the second and third electrodes being operable as recording or sensing electrodes configured and located sufficiently close to the first electrode to permit differential sensing and measurement of complex electrical impedance signals, including the phase and amplitude thereof, resulting from injection of the constant current AC signals into the cardiac tissue from the first electrode, wherein one or more of the plurality of electrodes are further configured to deliver PFA energy into, and to ablate, the patient's cardiac tissue.

2. The intracardiac PFA system of claim 1, wherein electrical signals corresponding to transmitted controlled constant current AC signals sensed by the second and third electrodes as complex electrical impedance electrical signals are provided or relayed to the data acquisition, recording, or measurement device.

3. The intracardiac PFA system of claim 1, wherein the data acquisition, recording, or measurement devices are configured to relay the sensed electrical signals to the at least one computing device as sensed electrical signal values.

4. The intracardiac PFA system of claim 3, wherein the at least one computing device comprises at least one non-transitory computer readable medium configured to store instructions executable by at least one processor to permit detection or display to a user of changes in the amplitude and phase of the sensed electrical signals or values that correspond to a condition or state of the cardiac tissue that has been subjected to the PFA energy.

5. The intracardiac PFA system of claim 4, wherein the detected or displayed condition or state of the cardiac tissue is live tissue, dead tissue, or a combination of live and dead tissue.

6. The intracardiac PFA system of claim 4, wherein the detected or displayed condition or state of the cardiac tissue is reversibly electroporated tissue, irreversibly electroporated tissue, or a combination of reversibly electroporated tissue and irreversibly electroporated tissue.

7. The intracardiac PFA system of claim 4, wherein changes in the amplitude and phase of the sensed electrical signals or values are based upon comparisons between sensed electrical signals or values derived from the second electrode and sensed electrical signals or values derived from the third electrode.

8. The intracardiac PFA system of claim 1, wherein a distance between the first electrode and the second electrode, or between the first electrode and the third electrode, ranges between about 0.25 cm and about 4 cm.

9. The intracardiac PFA system of claim 1, wherein a distance between the first electrode and the second electrode, or between the first electrode and the third electrode, ranges between about 0.5 cm and about 2 cm.

10. The intracardiac PFA system of claim 1, wherein the constant current AC signals have a frequency ranging between about 10 kHz and about 500 kHz.

11. The intracardiac PFA system of claim 1, wherein the constant current AC signals have a frequency ranging between about 50 kHz and about 150 KHz.

12. The intracardiac PFA system of claim 1, wherein the constant current AC signals have a current ranging between about 0.4 mA and about 3 mA.

13. The intracardiac PFA system of claim 1, wherein the constant current AC signals have a current ranging between about 0.5 mA and about 1.5 mA.

14. The intracardiac PFA system of claim 1, wherein one or more of the polarity and functionality of at least one of the current injection electrode, the sensing electrodes, and the ablation electrode can be interchanged or switched.

15. The intracardiac PFA system of claim 1, further comprising a PFA control device configured to controllably provide pulsed field energy to one or more of the plurality of electrodes.

16. An intracardiac method of sensing, recording and analyzing complex electrical impedance signals using an intracardiac pulse field ablation (PFA) catheter located inside a patient's heart, the PFA catheter comprising a proximal end and a distal end, a catheter body located between the proximal end and the distal end, and a plurality of electrodes including at least first, second and third electrodes, the PFA catheter and the electrodes thereof being configured to be operably connected to a data acquisition, recording or measurement device, a constant current AC signal generator, and at least one computing device, the first, second, third and fourth electrodes being located near or in the direction of the distal end, the first electrode being operable as a signal injection electrode configured to inject constant current AC signals into or near the patient's cardiac tissue, the second and third electrodes being operable as recording or sensing electrodes configured and located sufficiently close to the first electrode to permit differential sensing and measurement of complex electrical impedance signals, including the phase and amplitude thereof, resulting from injection of the constant current AC signals into the cardiac tissue from the first electrode, wherein one or more of the plurality of electrodes are further configured to deliver PFA energy into, and to ablate, the patient's cardiac tissue, the method comprising:
positioning the PFA catheter inside the patient's heart so that the fourth ablation electrode is located near a target for cardiac tissue ablation;
using the fourth ablation electrode, applying pulsed field ablation energy to the target cardiac tissue;
using the first current injection electrode, injecting constant current AC signals into or near the target cardiac tissue;
using the second and third recording or sensing electrodes, differentially sensing and measuring complex electrical impedance signals resulting from injection of the constant current AC signals into the cardiac tissue from the first electrode; and
relaying the differentially sensed complex electrical impedance signals to the at least one computing device as sensed electrical signal values.

17. The method of claim 16, wherein the at least one computing device comprises at least one non-transitory computer readable medium configured to store instructions executable by at least one processor to permit detection or display to a user changes in the amplitude and phase of the sensed electrical signals or values that correspond to a condition or state of the cardiac tissue that has been subjected to the PFA energy, and further comprising at least one of detecting and displaying to the user changes in the amplitude and phase of the sensed electrical signals or values that correspond to a condition or state of the cardiac tissue that has been subjected to the PFA energy.

18. The method of claim 17, wherein changes in the amplitude and phase of the sensed electrical signals or values are based upon comparisons between sensed electrical signals or values derived from the second electrode and sensed electrical signals or values derived from the third electrode.

19. The method of claim 18, wherein the constant current AC signals have a frequency ranging between about 50 kHz and about 150 kHz.

20. The method of claim 19, wherein the constant current AC signals have a current ranging between about 0.5 mA and about 1.5 mA.
